(19) United States
(12) Patent Application Publication
(10) Pub. No.: US 2010/0021456 A1
(43) Pub. Date: Jan. 28, 2010

(54) DRUG FOR INHIBITING,PREVENTING OR TREATMENT OF RHEUMATOID ARTHRITIS

(75) Inventors: Pierre Miossec, Bron (FR); Ling Toh, Lyon (FR); Saloua Zrioual, Lyon (FR)

Correspondence Address: OLIFF & BERRIDGE, PLC, P.O. BOX 320850, ALEXANDRIA, VA, 22320-4850, US

(73) Assignees: BIOMERIEUX, Marcy L'etoile (FR); HOSPICES CIVILS DE LYON, Lyon (FR)

(21) Appl. No.: 12/312,375

(22) PCT Filed: Nov. 28, 2007
(86) PCT No.: PCT/FR2007/052409

§ 371 Date: May 7, 2009

(30) Foreign Application Priority Data
Nov. 29, 2006 (FR) 0655194

(51) Int. Cl. A61K 39/395; A61K 38/00; A61K 48/00; A61P 19/02; G01N 33/53

(52) U.S. Cl. 424/133.1; 514/2; 424/141.1; 424/158.1; 514/44 A; 435/6

(57) ABSTRACT

The present invention relates to the use of at least one interleukin-17F inhibitor and/or of at least one IL-17 receptor inhibitor, for the manufacture of a medicament for inhibiting, preventing or treating rheumatoid arthritis.

The invention also relates to a pharmaceutical composition comprising, as active ingredient, at least one interleukin-17F inhibitor and/or at least one IL-17 receptor inhibitor in combination with a pharmaceutically appropriate carrier, and also to the use thereof for inhibiting, preventing or treating rheumatoid arthritis.

The present invention relates to rheumatoid arthritis, and in particular to the use of at least one interleukin-17F inhibitor and/or of at least one IL-17 receptor inhibitor, for the manufacture of a medicament for inhibiting, preventing or treating rheumatoid arthritis. The invention also relates to a pharmaceutical composition comprising, as active ingredient, at least one interleukin-17F inhibitor and/or at least one IL-17 receptor inhibitor in combination with a pharmaceutically appropriate carrier, and also to the use thereof for inhibiting, preventing or treating rheumatoid arthritis. Rheumatoid arthritis (RA) is a chronic condition, which is characterized by the inflammation, and deformation of several joints with, in addition, a risk of extra-articular complications. Knowledge concerning the role of cytokines in cell-cell interactions has led to the reasonable development of treatments with anticytokine agents.

The seriousness of the condition varies from one individual to the other, but it is associated, in the long-term, with an increase in morbidity and in mortality. Symptomatic treatment makes use of nonsteroidal anti-inflammatories, and optionally corticosteroids. Currently, methotrexate appears to be the reference treatment. Treatments which inhibit proinflammatory cytokines are also proposed in combination with DMARDs for the maintenance treatment of rheumatoid arthritis. In this respect, mention may be made of etanercept and infliximab, which are two inhibitors directed against TNF (tumor necrosis factor), a cytokine involved in the inflammatory process of rheumatoid arthritis. Such molecules are described as anti-TNF. More generally, the TNF-alpha factor has emerged as a main therapeutic target based on two clinical studies with biological inhibitors such as monoclonal antibodies or soluble receptors. In this respect, Infliximab® is prescribed for reducing inflammation, but also for slowing down the development of rheumatoid arthritis when other medicaments are insufficient.

Mention may also be made of other treatments, which implement the use of antagonists of interleukins (IL), such as IL-1 (Anakinra), CTLA4Ig (blocking of CD80-86), anti-IL-6 receptor monoclonal antibodies (MRA), anti-CD20 antibodies (Rituximab).

The progressive nature of the RA can be determined using several elements: pain, inflammation, joint mobility, functional impotence, quality of life, or lifespan. Clinically, the study of the progressive nature of the response over the course of a treatment is based on the use of standardized response indices which integrate the elements mentioned above, and among which the DAS (disease activity score) criterion, or variants thereof (Van der Heijde D. M. et al., J Rheumatol, 1993, 20(3): 579-81; Prevoo M. L. et al, Arthritis Rheum, 1995, 38: 44-8) and the ACR criterion (Felson D. T. et al, 1993, Arthritis Rheum, 36: 729-40) are the most widely used.

Thus, the current treatments have limits since approximately 30% of patients do not respond to the biotherapies. Furthermore, whatever the treatment, the disease remains recurrent. For example, not all patients respond in a comparable manner to treatment with Infliximab®. Thus, X-rays of joints of patients suffering from rheumatoid arthritis and treated with Infliximab®, taken after one year, have revealed that, although a large number of patients benefited from an improvement, a smaller number had experienced joint deterioration.

It is therefore important, from a clinical point of view, to propose new treatment alternatives to the clinician. In addition, it is also important, from a clinical point of view, to determine, before any prescription, whether or not the patient responds to the treatment proposed by the physician.

The present invention proposes to solve the drawbacks of the prior art by providing new biological tools for improving the treatment of a patient for rheumatoid arthritis. The present invention in fact provides new medicaments for improving the treatment of a patient for rheumatoid arthritis. Furthermore, the present invention makes it possible to determine the response of a patient suffering from rheumatoid arthritis to a treatment such as Infliximab®. The present invention is also very relevant for monitoring the response of a patient subjected to a treatment such as Infliximab®.

The inventors have demonstrated that interleukin-17F (IL-17F), just like interleukin-17A (IL-17A), plays a very important role in the physiology of rheumatoid arthritis, and that blocking the IL-17 receptors A and C potentiates the effect of an anti-TNF-alpha treatment.

In this respect, the invention concerns the use of at least one interleukin-17F inhibitor and/or of at least one IL-17 receptor inhibitor, for the manufacture of a medicament for inhibiting, preventing or treating rheumatoid arthritis.

The invention also concerns the use of at least one interleukin-17F inhibitor and/or of at least one IL-17 receptor inhibitor in combination with a treatment against TNF-alpha, for the manufacture of a medicament for inhibiting, preventing or treating rheumatoid arthritis.

Preferably, the invention concerns the use of at least one IL-17 receptor A inhibitor and/or of at least one IL-17 receptor C inhibitor.

The invention also concerns a pharmaceutical composition comprising, as active ingredient, at least one interleukin-17F inhibitor and/or at least one IL-17 receptor inhibitor in combination with a pharmaceutically appropriate carrier, and also the use of such a composition for inhibiting, preventing or treating rheumatoid arthritis.

Preferably, the composition also comprises a treatment against TNF-alpha.

Preferably, the composition comprises at least one IL-17 receptor A inhibitor and at least one IL-17 receptor C inhibitor.

Preferably, said IL-17 receptor is the IL-17 receptor A or the IL-17 receptor C.

Preferably, said treatment against TNF-alpha is chosen from etanercept, Infliximab® and adalimumab, and even more preferably etanercept.

According to one particular embodiment, said treatment against TNF-alpha is in combination with a cytostatic compound which inhibits cell proliferation, such as methotrexate.

Preferably, the interleukin-17F inhibitor is an antibody directed against IL-17F, and/or the inhibitor of said IL-17 receptor is an antibody directed against the IL-17 receptor, preferably against the IL-17 receptor A or C.

Preferably, the interleukin-17F inhibitor is an interfering RNA against IL-17F, and/or the inhibitor of said IL-17 receptor is an interfering RNA against the IL-17 receptor, preferably against the IL-17 receptor A or C.

The following definitions will make it possible to understand the invention more clearly.

The term “interleukin-17F inhibitor” is intended to mean a molecule (or a collection of molecules) which block(s) the inflammatory and/or immunostimulant activity of IL-17F.

The inhibitor may in particular be an antibody directed against IL-17F. The term “antibody” is intended to mean both the whole antibody and an antibody fragment.

The recombinant antibodies can be obtained according to conventional methods known to those skilled in the art, using prokaryotic organisms, such as bacteria, or using eukaryotic organisms, such as yeasts, mammalian cells, plant cells, insect cells or animal cells, or by means of extracellular production systems.

The monoclonal antibodies may be prepared according to the conventional techniques known to those skilled in the art, such as the hybridoma technique, the general principle of which is summarized below.

Firstly, an animal, generally a mouse (or cells in culture in the case of in vitro immunizations) is immunized with a target antigen of interest, and the B lymphocytes of said animal are then capable of producing antibodies against said antigen. These antibody-producing lymphocytes are subsequently fused with “immortal” myeloma cells (murine in the example) so as to produce hybridomas. Using the heterogeneous cell mixture thus obtained, a selection of the cells capable of producing a particular antibody and of multiplying indefinitely is then carried out. Each hybridoma is multiplied in the form of a clone, each resulting in the production of a monoclonal antibody of which the recognition properties with respect to the antigen of interest may be tested, for example, by ELISA, by one- or two-dimensional immunoblotting, by immunofluorescence, or using a biosensor. The monoclonal antibodies thus selected are subsequently purified, in particular according to the affinity chromatography technique.

Antibody fragments can, for example, be obtained by proteolysis. Thus, they can be obtained by enzymatic digestion, resulting in fragments of Fab type (treatment with papain; Porter R R, 1959, Biochem. J., 73: 119-126) or of F(ab)′2 type (treatment with pepsin; Nisonoff A. et al., 1960, Science, 132: 1770-1771). They can also be prepared by the recombinant process (Skerra A., 1993, Curr. Opin. Immunol., 5: 256-262). Another antibody fragment which is suitable for the purposes of the invention comprises an Fv fragment, which is a dimer constituted of the noncovalent association of the variable light (VL) domain and of the variable heavy (VH) domain of the Fab fragment, and therefore the association of two polypeptide chains. In order to improve the stability of the Fv fragment due to the dissociation of the two polypeptide chains, this Fv fragment can be modified by genetic engineering, by inserting a suitable peptide linker between the VL domain and the VH domain (Huston P. et al., 1988, Proc. Natl. Acad. Sci. USA, 85: 5879-5883). This is then referred to as an scFv fragment (single chain fragment variable) since it is made up of a single polypeptide chain. The use of a peptide linker composed preferentially of 15 to 25 amino acids makes it possible to link the C-terminal end of one domain to the N-terminal end of the other domain, thus constituting a monomeric molecule having binding properties similar to those of the antibody in its complete form. Both orientations of the VL and VH domain are suitable (VL-linker-VH and VH-linker-VL) since they have identical functional properties. Of course, any fragment known to those skilled in the art and having the immunological characteristics defined above is suitable for the purposes of the invention.

The inhibitor may also be an interfering RNA against IL-17F.

The term “interfering RNA” is intended to mean a ribonucleic acid which blocks the expression of a predetermined gene (Dallas A. et al., 2006, Med Sci Monit, 12(4): RA67-74).

The term “IL-17 receptor” is intended to mean a molecule of the IL-17 receptor family, said receptors being defined by their likeness to the IL-17RA receptor (Moseley T. A. et al., 2003, Cytokine Growth Factor Rev, 14(2): 155-74).

The term “IL-17RA receptor” is intended to mean the molecule initially discovered for its involvement in the inflammatory and/or immunostimulant activity of IL-17A (Yao Z. et al., 1997, Cytokine, 9(11): 794-800).

The term “IL-17RC receptor” is intended to mean an IL-17RA-receptor-like molecule (Haudenschild D. et al., 2002, J Biol Chem, 277: 4309-4316).

The term “IL-17 receptor inhibitor” is intended to mean a molecule which blocks the action of an IL-17 receptor.

The inhibitor may in particular be an antibody, as defined above, directed against the IL-17 receptor, preferably against the IL-17 receptor A or C.

The inhibitor may also be an interfering RNA, as defined above, against the IL-17 receptor, preferably against the IL-17 receptor A or C.

The term “treatment against TNF-alpha” (or anti-TNF-alpha treatment) is intended to mean a treatment, a compound or a medicament which blocks the action of TNF (tumor necrosis factor), such as, in particular, infliximab, etanercept and adalimumab.

The term “medicament” or “pharmaceutical composition” is intended to mean any substance or composition presented as having curative or preventive properties with regard to human or animal diseases, and also any product that can be administered to humans or to animals with a view to establishing a medical diagnosis or to restoring, correcting or modifying their organic functions.

The term “active substance” is intended to mean a compound acknowledged as having therapeutic properties.

In the pharmaceutical compositions according to the invention, for oral, sublingual, subcutaneous, intramuscular, intravenous, topical, intratracheal, rectal or transdermal administration, the active substances may be administered in unit administration forms or as a mixture with conventional carriers, which are intended for oral administration, for example in the form of a tablet, a gel capsule, an oral solution, etc, or rectal administration, in the form of a suppository, parenteral administration, in particular in the form of an injectable solution, especially intravenous, intradermal, subcutaneous, etc., administration, according to conventional protocols well known to those skilled in the art. For topical application, the active substances can be used in creams, ointments or lotions.

When a solid composition in tablet form is prepared, the active substances are mixed with a pharmaceutically acceptable excipient, also known as suitable pharmaceutical carrier, such as gelatin, starch, lactose, magnesium stearate, talc, gum arabic, or the like. The tablets can be coated with sucrose, a cellulosic derivative, or other suitable substances. They can also be treated in such a way that they have a sustained or delayed activity and that they continuously release a predetermined amount of active substances. It is also possible to obtain a preparation of gel capsules by mixing the active substances with a diluent and by pouring the mixture into soft or hard gel capsules. It is also possible to obtain a preparation in syrup form or for administration in the form of drops, in which the active substances are present together with a sweetener, an antiseptic, for instance methylparaben and propylparaben, and also an enhancer or a suitable dye. Water-dispersible powders or granules can contain the active ingredients as a mixture with dispersing agents or wetting agents, or suspending agents, well known to those skilled in the art. For parenteral administration, use is made of isotonic saline solutions or injectable sterile solutions which contain dispersing agents, and pharmacologically compatible wetting agents, such as in particular propylene glycol or butylene glycol.

The medicament or the pharmaceutical composition according to the invention may also comprise an activating agent which induces the effects of a medication or reinforces or supplements the effects of the principle medication, by increasing in particular the bioavailability of the principle medication.

The posology depends on the seriousness of the condition. In the case of a pharmaceutical composition comprising an antibody, the administration may in particular, be carried out once every 2 to 8 weeks, preferably with 50 to 100 mg of antibody, in combination with a pharmaceutically acceptable excipient. In the case of a pharmaceutical composition comprising an interfering RNA, the administration may in particular be carried out once every 2 to 8 weeks, preferably with 1 to 10 mg/Kg of interfering RNA, in combination with a pharmaceutically acceptable excipient.

The invention also concerns an in vitro method for determining, on the basis of a biological sample,
the early diagnosis of rheumatoid arthritis,
the response of a patient suffering from rheumatoid arthritis to a treatment directed against a cytokine involved in the inflammatory process of the disease,
the monitoring of the response of a patient suffering from rheumatoid arthritis to a treatment directed against a cytokine involved in the inflammatory process of the disease over time,
   characterized in that the expression of the gene encoding IL-17A, IL-17F, IL-17RA and/or IL-17RC is determined.

The measurement of the expression of the gene encoding IL-17A, IL-17F, IL-17RA and/or IL-17RC comprises the following steps:
a) biological material is extracted from the biological sample;
b) the biological material is brought into contact with at least one reagent specific for the gene encoding IL-17A, IL-17F, IL-17RA and/or IL-17RC;
c) the expression of the gene encoding IL-17A, IL-17F, IL-17RA and/or IL-17RC is determined.

The biological material extracted during step a) may comprise nucleic acids or proteins.

Said specific reagent of step b) may comprise a hybridization probe or an antibody specific for the gene encoding IL-17A, IL-17F, IL-17RA and/or IL-17RC.

For the purpose of the present invention, the term “biological sample” is intended to mean any sample taken from a patient, and liable to contain a biological material as defined hereinafter. This biological sample may in particular be a sample of blood, serum, tissue, synovial fluid or synoviocytes from the patient. This biological sample is obtained by any sampling means known to those skilled in the art. According to one preferred embodiment of the invention, the biological sample taken from the patient is a blood sample.

During step a) of the method according to the invention, the biological material is extracted from the biological sample by any of the protocols for extracting and purifying nucleic acids or proteins known to those skilled in the art.

For the purpose of the present invention, the term “biological material” is intended to mean any material which makes it possible to detect the expression of a target gene. The biological material may comprise in particular proteins, or nucleic acids such as, in particular, deoxyribonucleic acids (DNA) or ribonucleic acids (RNA). The nucleic acid may in particular be an RNA (ribonucleic acid). According to one preferred embodiment of the invention, the biological material extracted during step a) comprises nucleic acids, preferably RNAs, and even more preferably total RNA. Total RNA comprises transfer RNAs, messenger RNAs (mRNAs), such as the mRNAs transcribed from the target gene, but also transcribed from any other gene, and ribosomal RNAs. This biological material comprises material specific for a target gene, such as, in particular, the mRNAs transcribed from the target gene or the proteins derived from these mRNAs, but may also comprise material not specific for a target gene, such as, in particular, the mRNAs transcribed from a gene other than the target gene, the tRNAs, or the rRNAs derived from genes other than the target gene.

By way of indication, the nucleic acid extraction can be carried out by means of:
a step of lysis of the cells present in the biological sample, in order to release the nucleic acids contained in the patient's cells. By way of example, use may be made of the lysis methods as described in patent applications WO 00/05338, WO 99/53304 and WO 99/15321. Those skilled in the art may use other well-known methods of lysis, such as heat shock or osmotic shock or chemical lysis with chaotropic agents such as guanidium salts (U.S. Pat. No. 5,234,809);
a step of purification, for separating the nucleic acids from the other cell constituents released in the lysis step. This step generally makes it possible to concentrate the nucleic acids, and can be adapted to the purification of DNA or of RNA. By way of example, it is possible to use magnetic particles optionally coated with oligonucleotides, by adsorption or covalence (in this respect, see patents U.S. Pat. No. 4,672,040 and U.S. Pat. No. 5,750,338), and thus to purify the nucleic acids which are bound to these magnetic particles, by means of a washing step. This nucleic acid purification step is particularly advantageous if it is desired to subsequently amplify said nucleic acids. One particularly advantageous embodiment of these magnetic particles is described in patent applications: WO-A-97/45202 and WO-A-99/35500. Another advantageous example of a nucleic acid purification method is the use of silica, either in the form of a column, or in the form of inert or magnetic particles. Other, very widely used, methods are based on ion exchange resins in a column or in a paramagnetic particulate format. Another very relevant but nonexclusive method for the invention is that of adsorption onto a metal oxide support.

In the case of the extraction of proteins, the first step generally comprises, as for the nucleic acids, lysis of the cells. An osmotic shock may be sufficient to rupture the cell membrane of fragile cells, it being possible for said osmotic shock to be carried out in the presence of a detergent. A mechanic action may also be added to the process (piston homogenizer, for example). The lysis may also be induced by ultrasound, or by mechanical lysis using glass beads. The extraction of the proteins of interest can subsequently be carried out by chromatography, such as, in particular, on a gel chromatography column, packed with a resin comprising hollow, porous beads. The pore size of these beads is such that the proteins are separated according their size. Mention may also be made of ion exchange column chromatography, which enables proteins to be extracted according to their electrostatic affinity with respect to charge groups of the resin.

During step b), and for the purpose of the present invention, the term “specific reagent” is intended to mean a reagent which, when it is brought into contact with biological material as defined above, binds with the materials specific for said target gene.

By way of indication, when the specific reagent and the biological material are of nucleic origin, bringing the specific reagent into contact with the biological material enables the specific reagent to hybridize with the material specific for the target gene. The term “hybridization” is intended to mean the process during which, under suitable conditions, two nucleotide fragments bind to one another with stable, specific hydrogen bonds so as to form a double-stranded complex. These hydrogen bonds form between the complementary bases adenine (A) and thymine (T) (or uracil (U)) (this is then referred to as an A-T bond) or between the complementary bases guanine (G) and cytosine (C) (this is then referred to as a G-C bond). The hybridization of two nucleotide fragments may be total (reference is then made to complementary nucleotide fragments or sequences), i.e. the double-stranded complex obtained during this hybridization comprises only A-T bonds and C-G bonds. This hybridization may be partial (reference is then made to sufficiently complementary nucleotide fragments or sequences), i.e. the double-stranded complex obtained comprises A-T bonds and C-G bonds allowing the double-stranded complex to form, but also bases not bonded to a complementary base. The hybridization between two nucleotide fragments depends on the working conditions which are used, and in particular on the stringency. The stringency is defined in particular according to the base composition of the two nucleotide fragments, and also by the degree of mismatching between two nucleotide fragments. The stringency may also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art. In general, according to the length of the nucleotide fragments that it is desired to hybridize, the hybridization temperature is between approximately 20 and 70° C., in particular between 35 and 65° C., in a saline solution at a concentration of approximately 0.5 to 1 M. A sequence, or nucleotide fragment, or oligonucleotide, or polynucleotide, is a series of nucleotide motifs assembled together by phosphoric ester bonds, characterized by the informational sequence of the natural nucleic acids, capable of hybridizing to a nucleotide fragment, it being possible for the series to contain monomers of different structures and to be obtained from a natural nucleic acid molecule and/or by genetic recombination and/or by chemical synthesis. A motif is derived from a monomer which may be a natural nucleotide of a nucleic acid, the constitutive elements of which are a sugar, a phosphate group and a nitrogenous base; in DNA, the sugar is deoxy-2-ribose, in RNA, the sugar is ribose; depending on whether it is a question of DNA or RNA, the nitrogenous base is chosen from adenine, guanine, uracil, cytosine and thymine; or alternatively the monomer is a nucleotide modified in at least one of the three constitutive elements; by way of example, the modification may occur either at the level of the bases, with modified bases such as inosine, methyl-5-deoxycytidine, deoxyuridine, dimethylamino-5-deoxyuridine, diamino-2,6-purine, bromo-5-deoxyuridine or any other modified base capable of hybridization, or at the level of the sugar, for example the replacement of at least one deoxyribose with a polyamide, or else at the level of the phosphate group, for example replacement thereof with esters chosen in particular from diphosphates, alkyl and aryl phosphonates and phosphorothioates.

According to one particular embodiment of the invention, the specific reagent comprises at least one amplification primer. For the purpose of the present invention, the term “amplification primer” is intended to mean a nucleotide fragment comprising from 5 to 100 nucleic motifs, preferably from 15 to 30 nucleic motifs, allowing the initiation of an enzymatic polymerization, such as, in particular, an enzymatic amplification reaction. The term “enzymatic amplification reaction” is intended to mean a process generating multiple copies of a nucleotide fragment through the action of at least one enzyme. Such amplification reactions are well known to those skilled in the art and mention may in particular be made of the following techniques:
PCR (Polymerase Chain Reaction), as described in patents U.S. Pat. No. 4,683,195, U.S. Pat. No. 4,683,202 and U.S. Pat. No. 4,800,159,
LCR (Ligase Chain Reaction), disclosed, for example, in patent application EP 0 201 184,
RCR (Repair Chain Reaction), described in patent application WO 90/01069,
3SR (Self Sustained Sequence Replication) with patent application WO 90/06995,
NASBA (Nucleic Acid Sequence-Based Amplification) with patent application WO 91/02818, and
TMA (Transcription Mediated Amplification) with patent U.S. Pat. No. 5,399,491.

When the enzymatic amplification is a PCR, the specific reagent comprises at least 2 amplification primers, specific for a target gene, in order to allow the amplification of the target-gene-specific material. The target-gene-specific material then preferably comprises a complementary DNA obtained by reverse transcription of messenger RNA derived from the target gene (reference is then made to target-gene-specific cDNA) or a complementary RNA obtained by transcription of the cDNAs specific for a target gene (reference is then made to target-gene-specific cRNA). When the enzymatic amplification is a PCR carried out after a reverse transcription reaction, this is referred to as RT-PCR.

According to another preferred embodiment of the invention, the specific reagent of step b) comprises at least one hybridization probe.

The term “hybridization probe” is intended to mean a nucleotide fragment comprising at least 5 nucleotide motifs, for instance from 5 to 100 nucleic motifs, in particular from 10 to 35 nucleic motifs, having a hybridization specificity under given conditions so as to form a hybridization complex with the material specific for a target gene. In the present invention, the target-gene-specific material may be a nucleotide sequence included in a messenger RNA derived from the target gene (reference is then made to target-gene-specific mRNA), a nucleotide sequence included in a complementary DNA obtained by reverse transcription of said messenger RNA (reference is then made to target-gene-specific cDNA), or else a nucleotide sequence included in a complementary RNA obtained by transcription of said cDNA as described above (reference will then be made to target-gene-specific cRNA). The hybridization probe may comprise a label for its detection. The term “detection” is intended to mean either a direct detection by a physical method, or an indirect detection by a detection method using a label. Many detection methods exist for detecting nucleic acids [see, for example, Kricka et al., Clinical Chemistry, 1999, No. 45(4), p. 453-458 or Keller G. H. et al., DNA Probes, 2nd Ed., Stockton Press, 1993, sections 5 and 6, p. 173-249]. The term “label” is intended to mean a tracer capable of generating a signal that can be detected. A nonlimiting list of these tracers includes enzymes which produce a signal detectable, for example, by colorimetry, fluorescence or luminescence, such as horseradish peroxydase, alkaline phosphatase, beta-galactosidase, glucose-6-phosphate dehydrogenase; chromophores such as fluorescent, luminescent or dye compounds; electron dense groups detectable by electron microscopy or by their electrical properties such as conductivity, by amperometry or voltametry methods, or by impedance measurements; groups that can be detected by optical methods such as diffraction, surface plasmon resonance, contact angle variation or by physical methods such as atomic force spectroscopy, tunnel effect, etc.; radioactive molecules such as ³²P, ³⁵S or ¹²⁵I.

For the purpose of the present invention, the hybridization probe may be a “detection” probe. In this case, the “detection” probe is labeled with a label as defined above. The detection probe may in particular be a “molecular beacon” detection probe as described by Tyagi & Kramer (Nature biotech, 1996, 14:303-308). These “molecular beacons” become fluorescent during hybridization. They have a stem-loop structure and contain a fluorophore and a quencher group. The binding of the specific loop sequence with its complementary target nucleic acid sequence causes the stem to uncoil and a fluorescent signal to be emitted during excitation at the appropriate wavelength.

For the detection of the hybridization reaction, use may be made of target sequences that have been labeled directly (in particular by the incorporation of a label within the target sequence) or indirectly (in particular using a detection probe as defined above) the target sequence. A step for labeling and/or cleaving the target sequence can in particular be carried out before the hybridization step, for example using a labeled deoxyribonucleotide triphosphate during the enzymatic amplification reaction. The cleavage can be carried out in particular through the action of imidazole and manganese chloride. The target sequence can also be labeled after the amplification step, for example by hybridizing a detection probe according to the sandwich hybridization technique described in document WO 91/19812. Another particular preferred method for labeling nucleic acids is described in application FR 2 780 059. According to one preferred embodiment of the invention, the detection probe comprises a fluorophore and a quencher.

The hybridization probe may also be a “capture” probe. In this case, the “capture” probe is immobilized or immobilizable on a solid support by any appropriate means, i.e. directly or indirectly, for example by covalence or adsorption. As solid support, use may be made of synthetic materials or natural materials, optionally chemically modified, in particular polysaccharides such as cellulose-based materials, for example paper, cellulose derivatives such as cellulose acetate and nitrocellulose, or dextran, polymers, copolymers, in particular based on styrene-type monomers, natural fibers such as cotton, and synthetic fibers such as nylon; mineral materials such as silica, quartz, glasses, ceramics; latices; magnetic particles; metal derivatives, gels, etc. The solid support may be in the form of a microtitration plate, of a membrane as described in application WO-A-94/12670, or of a particle. These steps of hybridization on a support may be preceded by an enzymatic amplification reaction step, as defined above, in order to increase the amount of target genetic material.

By way of indication, when the specific reagent and the biological material are of protein origin, bringing the specific reagent and the biological material into contact allows the formation of an “antigen-antibody” complex between the specific reagent and target-gene-specific material.

During step c), the determination of the expression of the target gene can be carried out by any of the protocols known to those skilled in the art.

In general, the expression of a target gene can be analyzed by detection of the mRNAs (messenger RNAs) which are transcribed from the target gene at a given instant or by the detection of the proteins derived from these mRNAs.

The invention preferentially concerns the determination of the expression of a target gene by detection of the mRNAs derived from this target gene.

When the specific reagent comprises one or more amplification primers, it is possible, during step c) of the method according to the invention, to determine the expression of a target gene in the following way:

1) after having extracted, as biological material, the total RNA (comprising the transfer RNAs (tRNAs), the ribosomal RNAs (rRNAs) and the messenger RNAs (mRNAs)) of a biological sample as presented above, a reverse transcription step is carried out in order to obtain the complementary DNAs (or cDNAs) of said mRNAs. By way of indication, this reverse transcription reaction can be carried out using a reverse transcriptase enzyme which makes it possible to obtain, from an RNA fragment, a complementary DNA fragment. The reverse transcriptase enzyme originating from AMV (Avian Myoblastosis Virus) or from MMLV (Moloney Murine Leukemia Virus) can in particular be used. When it is more particularly desired to obtain only the cDNAs of the mRNAs, this reverse transcription step is carried out in the presence of nucleotide fragments comprising only thymine bases (polyT), which hybridize by complementarity on the polyA sequence of the mRNAs so as to form a polyT-polyA complex which then serves as a starting point for the reverse transcription reaction carried out by the reverse transcriptase enzyme. cDNAs complementary to the mRNAs derived from a target gene (target-gene-specific cDNA) and cDNAs complementary to the mRNAs derived from genes other than the target gene (cDNAs not specific for the target gene) are then obtained;

2) the amplification primer(s) specific for a target gene is (are) brought into contact with the target-gene-specific cDNAs and the cDNAs not specific for the target gene. The amplification primer(s) specific for a target gene hybridize(s) with the target-gene-specific cDNAs and a predetermined region, of known length, of the cDNAs originating from the mRNAs derived from the target gene is specifically amplified. The cDNAs not specific for the target gene are not amplified, whereas a large amount of target-gene-specific cDNAs is then obtained. For the purpose of the present invention, reference is made, without distinction, to “target-gene-specific cDNAs” or to “cDNAs originating from the mRNAs derived from the target gene”. This step can be carried out in particular by a PCR-type amplification reaction or by any other amplification technique as defined above;

3) the expression of the target gene is determined by detecting and quantifying the target-gene-specific cDNAs obtained during step 2) above. This detection can be carried out after electrophoretic migration of the target-gene-specific cDNAs according to their size. The gel and the migration medium can include ethidium bromide so as to allow direct detection of the target-gene-specific cDNAs when the gel is placed, after a given migration period, on a UV (ultraviolet)-ray light table, through the emission of a light signal. The greater the amount of target-gene-specific cDNAs, the brighter this light signal. These electrophoresis techniques are well known to those skilled in the art. The target-gene-specific cDNAs can also be detected and quantified using a quantification range obtained by means of an amplification reaction carried out until saturation. In order to take into account the variability of enzymatic efficiency that may be observed during the various steps (reverse transcription, PCR, etc.), the expression of a target gene of various groups of patients can be standardized by simultaneously determining the expression of a “housekeeping” gene, the expression of which is similar in the various groups of patients. By realizing a ratio of the expression of the target gene to the expression of the housekeeping gene, i.e. by realizing a ratio of the amount of target-gene-specific cDNAs to the amount of housekeeping-gene-specific cDNAs, any variability between the various experiments is thus corrected. Those skilled in the art may refer in particular to the following publications: Bustin S A, J Mol Endocrinol, 2002, 29: 23-39; Giulietti A Methods, 2001, 25: 386-401.

When the specific reagent comprises at least one hybridization probe, the expression of a target gene can be determined in the following way:

1) after having extracted, as biological material, the total RNA of a biological sample as presented above, a reverse transcription step is carried out as described above in order to obtain cDNAs complementary to the mRNAs derived from a target gene (target-gene-specific cDNA) and cDNAs complementary to the mRNAs derived from genes other than the target gene (cDNA not specific for the target gene);

2) all the cDNAs are brought into contact with a support, on which are immobilized capture probes specific for the target gene whose expression it is desired to analyze, in order to carry out a hybridization reaction between the target-gene-specific cDNAs and the capture probes; the cDNAs not specific for the target gene do not hybridize to the capture probes. The hybridization reaction can be carried out on a solid support which includes all the materials as indicated above. According to one preferred embodiment, the hybridization probe is immobilized on a support. The hybridization reaction may be preceded by a step of enzymatic amplification of the target-gene-specific cDNAs, as described above, so as to obtain a large amount of target-gene-specific cDNAs and to increase the probability of a cDNA specific for a target gene hybridizing to a capture probe specific for the target gene. The hybridization reaction may also be preceded by a step for labeling and/or cleaving the target-gene-specific cDNAs, as described above, for example using a labeled deoxyribonucleotide triphosphate for the amplification reaction. The cleavage can be carried out in particular through the action of imidazole and manganese chloride. The target-gene-specific cDNA can also be labeled after the amplification step, for example by hybridizing a labeled probe according to the sandwich hybridization technique described in document WO-A-91/19812. Other particular preferred methods for labeling and/or cleaving nucleic acids are described in applications WO 99/65926, WO 01/44507, WO 01/44506, WO 02/090584 and WO 02/090319;

3) a step for detection of the hybridization reaction is subsequently carried out. The detection can be carried out by bringing the support, on which the target-gene-specific capture probes are hybridized with the target-gene-specific cDNAs, into contact with a “detection” probe labeled with a label, and detecting the signal emitted by the label. When the target-gene-specific cDNA has been labeled beforehand with a label, the signal emitted by the label is detected directly.

When the at least one specific reagent brought into contact in step b) of the method according to the invention comprises at least one hybridization probe, the expression of a target gene can also be determined in the following way:

1) after having extracted, as biological material, the total RNA of a biological sample as presented above, a reverse transcription step is carried out as described above in order to obtain the cDNAs of the mRNAs of the biological material. The polymerization of the complementary RNA of the cDNA is subsequently carried out using a T7 polymerase enzyme which functions under the control of a promoter and which makes it possible to obtain, from a DNA template, the complementary RNA. The cRNAs of the cDNAs of the mRNAs specific for the target gene (reference is then made to target-gene-specific cRNA) and the cRNAs of the cDNAs of the mRNAs not specific for the target gene are then obtained;

2) all the cRNAs are brought into contact with a support on which are immobilized capture probes specific for the target gene whose expression it is desired to analyze, in order to carry out a hybridization reaction between the target-gene-specific cRNAs and the capture probes; the cRNAs not specific for the target gene do not hybridize to the capture probes. The hybridization reaction can also be preceded by a step for labeling and/or cleaving the target-gene-specific cRNAs, as described above;

3) a step for detecting the hybridization reaction is subsequently carried out. The detection can be carried out by bringing the support, on which the target-gene-specific capture probes are hybridized with the target-gene-specific cRNAs, into contact with a “detection” probe labeled with a label, and detecting the signal emitted by the label. When the target-gene-specific cRNA has been labeled beforehand with a label, the signal emitted by the label is detected directly. The use of cRNA is particularly advantageous when a support of the biochip type on which a large number of probes are hybridized is used.

According to one particular embodiment of the invention, steps B and C are carried out at the same time. This preferred method can in particular be carried out by “real time NASBA”, which groups together, in a single step, the NASBA amplification technique and real time detection which uses “molecular beacons”. The NASBA reaction takes place in the tube, producing the single-stranded RNA with which the specific “molecular beacons” can simultaneously hybridize to give a fluorescent signal. The formation of the new RNA molecules is measured in real time by continuous verification of the signal in a fluorescent reader.

By way of indication, when the specific reagent and the biological material are of protein origin, step c) can in particular be carried out by Western blotting or ELISA, or any other method known to those skilled in the art.

By way of indication, the ELISA technique is a reference biochemical technique used in immunology for detecting the presence of an antibody or of an antigen in a sample. The technique uses two antibodies, one of them being specific to the antigen and the other being coupled to an enzyme.

By way of indication, the Western blotting technique is a test for detecting a specific protein in a sample using an antibody specific for this protein, comprising the following steps:

The first step is a gel of electrophoresis, which makes it possible to separate the proteins from the sample according to their size.

The proteins in the gel are then transferred onto a membrane (nitrocellulose, PVDF, etc.) by pressure or by application of an electric current, the proteins attaching to the membrane by virtue of hydrophobic and ionic interactions.

After saturation of the nonspecific interaction sites, a first antibody, specific for the protein to be studied (primary antibody), is incubated with the membrane.

The membrane is subsequently rinsed in order to remove the unbound primary antibodies, and then incubated with “secondary” antibodies, which will bind to the primary antibodies. This secondary antibody is normally bonded to an enzyme which allows visual identification of the protein studied on the membrane.

As for the ELISA techniques, the addition of a substrate for the enzyme generates a colored reaction which is visible on the membrane.

The invention also concerns the use of at least one reagent specific for the gene encoding IL-17A, IL-17F, IL-17RA and/or IL-17RC, for determining
the early diagnosis of rheumatoid arthritis;
the response of a patient suffering from rheumatoid arthritis to a treatment directed against a cytokine involved in the inflammatory process of the disease;
the monitoring of the response of a patient suffering from rheumatoid arthritis to a treatment directed against a cytokine involved in the inflammatory process of the disease, over time.

The invention also concerns a kit
for making an early diagnosis of rheumatoid arthritis;
for giving a prognosis for the response of a patient suffering from rheumatoid arthritis to a treatment directed against a cytokine involved in the inflammatory process of the disease;
for giving a prognosis for the monitoring of the response of a patient suffering from rheumatoid arthritis to a treatment directed against a cytokine involved in the inflammatory process of the disease, over time;
   comprising at least one reagent specific for the gene encoding IL-17A, IL-17F, IL-17RA and/or IL-17RC.

The analysis of the expression of the IL-17A, IL-17F, IL-17RA and/or IL-17RC genes then makes it possible to have a tool for the diagnosis/prognosis of the response of a patient suffering from rheumatoid arthritis to a treatment directed against a cytokine involved in the inflammatory process of the disease. It is, for example, possible to analyze the expression of the target gene in a patient whose reaction to a treatment directed against a cytokine involved in the inflammatory process of the disease is unknown, and to compare with known average expression values of the target gene of patients who respond to said treatment and known average expression values of the target gene of patients who do not respond to said treatment. This makes it possible to determine whether the patient is a responder or a nonresponder, which makes it possible to provide said patient with an appropriate treatment or to adapt his or her treatment throughout his or her therapy.

The figures will make it possible to understand the invention more clearly.

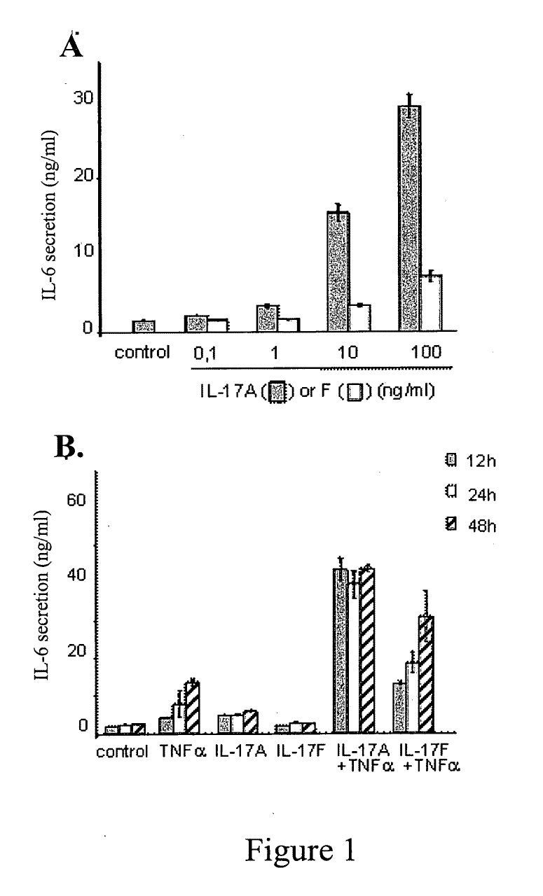

FIG. 1 represents the effect of IL-17A and IL-17F, alone or in combination with TNF-α, on IL-6 secretion. FIG. 1A represents the results obtained on synoviocytes of patients suffering from rheumatoid arthritis, RA, stimulated for 48 h at concentrations of IL-17A or IL-17F (0.1-100 ng/ml). FIG. 1B represents the results obtained on synoviocytes of patients suffering from rheumatoid arthritis, RA, stimulated for 12, 24 or 48 h with IL-17A and IL-17F (50 ng/ml), alone or in combination with TNF-α (0.5 ng/ml). The IL-6 was quantified by ELISA. The values represent the mean±SEM of results in triplicate. *=P<0.05 according to the Dunnett test. NS=not significant.

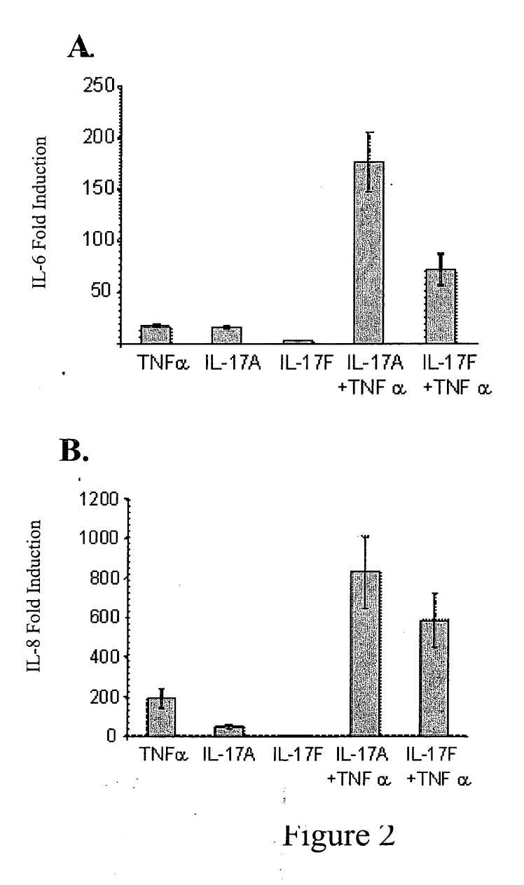

FIG. 2 represents the effects of IL-17A and IL-17F, alone or in combination with TNF-α, on the expression of messenger RNAs of proinflammatory mediators. The synoviocytes of patients suffering from rheumatoid arthritis, RA, were stimulated for 12 h with IL-17A or IL-17F (50 ng/ml), alone or in combination with TNF-α (0.5 ng/ml). The total RNA was extracted and reverse transcribed. The expression of the IL-6 mRNA (FIG. 2A) and the IL-8 mRNA (FIG. 2B) was quantified by real time RT-PCR. The values, standardized by the expression of GAPDH mRNA, were expressed by the ratio of the data obtained with RA synoviocytes to the data obtained under controlled conditions. The values represent the mean±SEM of results in quadruplicate. *=P<0.05 according to the Dunnett test. NS=not significant.

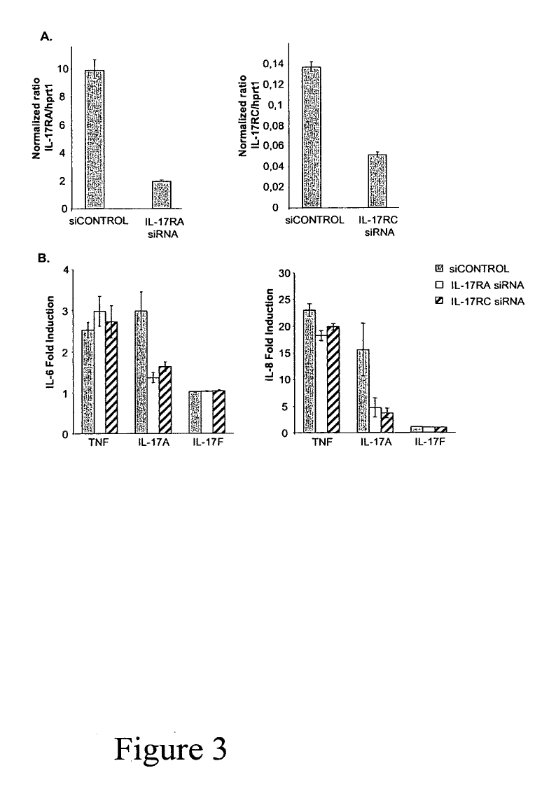

FIG. 3 represents the effects of IL-17RA iRNA and of IL-17RC iRNA on IL-6 secretion induced by IL-17A and IL-17F, by RA synoviocytes. The RA synoviocytes were transfected with IL-17RA iRNAs and IL-17RC iRNAs at, respectively, 0.5 and 0.005 μg. An siCONTROL iRNA was used as negative control. The effectiveness of the knockdown was studied by RT-PCR after 24 h and 48 h of transfection. FIG. 3A represents the expression of the IL-17RA mRNA and IL-17RC mRNA 24 h after transfection. The values represent the mean±SEM of results in triplicate. *=P<0.05 according to the Dunnett test. NS=not significant. FIG. 3B represents the results obtained 48 h after transfection, on RA synoviocytes transfected with siCONTROL RNA, IL-17RA siRNA or IL-17RC siRNA and stimulated for 12 h with IL-17A or IL-17F (50 ng/ml). The IL-6 was quantified by ELISA. The values represent the mean±SEM of results in triplicate. *=P<0.05 according to the Dunnett test. NS=not significant.

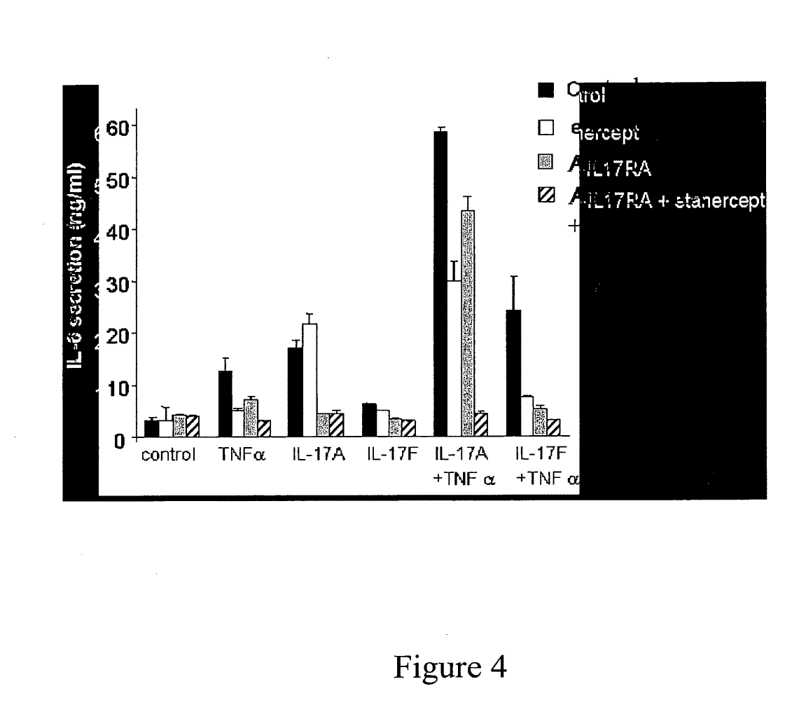

FIG. 4 represents the effects of the anti-IL-17RA antibodies on IL-6 secretion induced by IL-17A and IL-17F, and also the potentiating/beneficial effect of blocking IL-17RA in the presence of etanercept. The synoviocytes of patients suffering from rheumatoid arthritis, RA, were preincubated for 2 h (37° C., 5% CO₂) with various inhibitors: anti-IL-17RA antibody (10 μg/ml) alone or in combination with etanercept (10 μg/ml). The synoviocytes were stimulated with IL-17A or IL-17F (50 ng/ml), alone or in combination with TNFα (0.5 ng/ml). The IL-6 was quantified by ELISA. The values represent the mean±SEM of results in triplicate. *=P<0.05 according to the Dunnett test. NS=not significant.

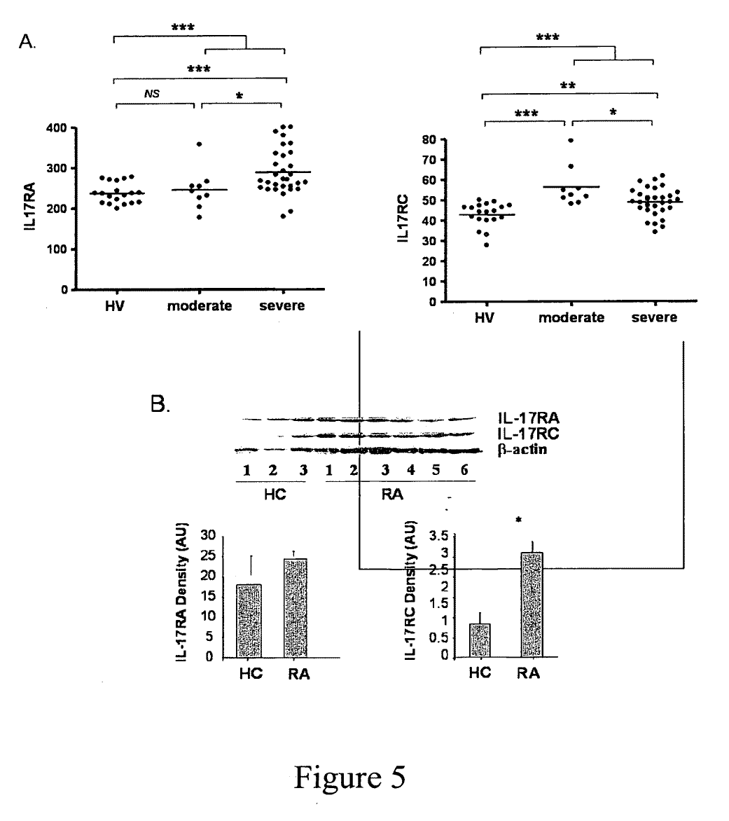

FIG. 5 represents the expression of IL-17RA and of IL-17RC in the blood of RA patients and of healthy volunteers (HV). FIG. 5A represents the expression of IL-17RA mRNA and IL-17RC mRNA in the peripheral blood of 40 RA patients (31 severe patients and 9 moderate patients) and 9 healthy volunteers, determined using DNA chips. The results are expressed as fluorescence intensity (P<0.05; **, P<0.005; ***P<0.0005 using the Mann-Whitney test). FIG. 5B represents the IL-17RA and IL-17RC protein expression in the peripheral blood of RA patients (n=6) and of healthy volunteers (n=3). The quantification was carried out by Western blotting. The densitometric data for IL-17RA expression and for IL-17RC expression was standardized using actin, and expressed in arbitrary units (AU) (*, P<0.05 by the Mann-Whitney test).

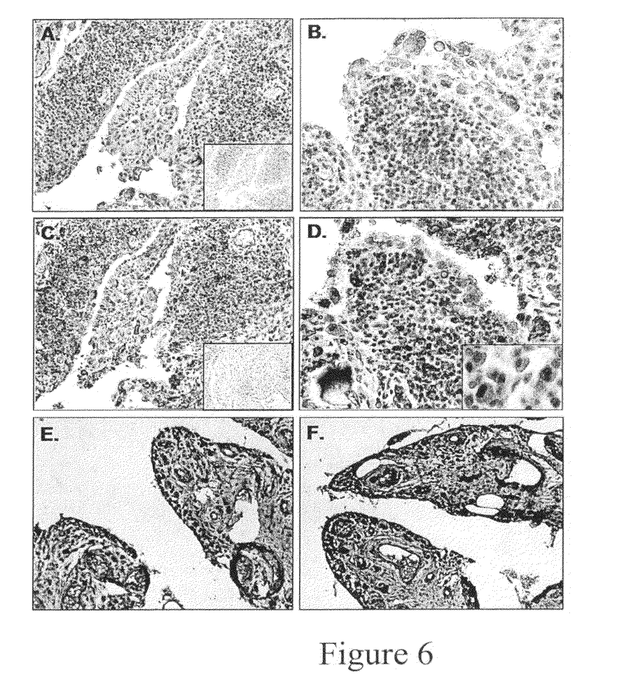

FIG. 6 represents the expression of IL-17RA and of IL-17RC in the synovial membrane. The immunohistochemical analysis was carried out on serial sections using murine anti-IL-17RA monoclonal antibodies (A and B, respectively×200 and×400) and goat anti-IL-17RC polyclonal antibodies (C and D, respectively×200 and×400). Control labelings were carried out with murine IgG1 and goat serum (×200) (insets in A and C, respectively). The immunodetection of IL-17RA and of IL-17RC was also carried out in arthritic synovial membrane (E and F, respectively; ×200). IL-17A expression was studied as a control (inset in D, ×600).

The following examples are given by way of illustration and are in no way limiting in nature. They will make it possible to understand the invention more clearly.

### MATERIALS & METHODS

A—Patients and healthy volunteers. 40 patients suffering from RA (RA) according to the ACR (American College of Rheumatology, 1987) criteria and 19 healthy volunteers (HV) were included in a study aimed at determining the gene expression profiles in the peripheral blood using U133A DNA chips (Affymetrix, UK Ltd). The clinical signs and the biological markers recorded include age, gender, duration of the disease, Larsen score, rheumatoid factor (RF), C-reactive protein (CRP) and the number of DMARDs. The patients were divided up into two groups depending on the Larsen score: destructive RA (Larsen score≧2), and nondestructive RA (Larsen score<2). On the basis of the DAS 28 (modified disease activity score (DAS) 28 joint index), 31 RA patients were evaluated as severe (DAS 28>3.2) and 9 as moderate (DAS28≦3.2). All the participants signed a written consent. The study protocol was approved by the Comité Consultatif de Protection des Personnes dans la Recherche Biomédicale (CCPPRB) [French Ethics Committee].

B—Cytokines and antibodies The human recombinant TNF-α used came from Sigma-Aldrich (St Louis, Mo.), while the recombinant human IL-17A and IL-17F proteins came from R&D Systems (Minneapolis, Minn.). The various antibodies used (monoclonal anti-IL-17RA antibody and polyclonal anti-IL-17RC antibodies) also came from R&D Systems. The soluble TNFRII receptor (etanercept) was provided by Wyeth (Louvain La Neuve, Belgium).

C—Cell culture The synoviocytes were obtained from synovial tissues derived from patients suffering from rheumatoid arthritis (RA synoviocytes) having undergone joint surgery, these patients meeting the criteria of the ACR (American College of Rheumatology). Briefly, the synovial tissues were cut up into small fragments and then incubated for 2 h at 37° C. in the presence of a mixture of proteolytic enzymes containing collagenase and hyaluronidase (Sigma-Aldrich) at 1 mg/ml. The resulting cells were cultured (37° C., 5% CO₂) in DMEM medium (Dulbecco's Modified Eagle's Medium, Invitrogen Life Technologies, Carlsbad, Calif.) supplemented with fetal calf serum (10% v/v), L-glutamine (2 mM) and a mixture of antibiotics (penicillin and streptomycin 100 U/ml). The various experiments were carried out with fibroblast-type rheumatoid synoviocytes, which correspond to the cells cultured for more than 3 passages.

D—interfering RNA (iRNA) A mixture of four iRNA duplexes (siGENOME SMARTPool siRNA) specific for IL-17RA (Genbank accession no.: NM_{—}014339) and for IL-17RC (Genbank accession No.: NM_{—}032732) was obtained from Dharmacon (Lafayette, Colo.). Dose-effect experiments were carried out in order to determine the smallest amount of iRNA necessary for a significant decrease in the levels of mRNA of interest (0.5 and 0.05 μg for the IL-17RA iRNAs and the IL-17RC iRNAs, respectively). RA synoviocytes were seeded at a cell density of 5×10⁵ per Petri dish (60 mm). The RA synoviocytes (70-80% confluence) were transfected with control iRNAs (siCONTROL iRNA as negative control and siGLO PPIB (cyclophilin B) iRNA as positive control) or with iRNAs of interest (IL-17RA SMARTPool iRNA and/or IL-17RC SMARTPool iRNA) by electroporation (Amaxa, Cologne, Germany) according to the working recommendations (reagents: Human Dermal Fibroblast Nucleofector, program U23). 48 h after transfection, the RA synoviocytes were stimulated for 12 h with IL-17A or IL-17F (50 ng/ml), alone or in combination with TNF-α (0.5 ng/ml). IL-6 and IL-8 were quantified in the culture supernatants by ELISA. The results of 3 independent experiments carried out with the siGENOME SMARTPool iRNAs were confirmed with the new ON-TARGETplus SMARTPool reagents iRNAs (Dharmacon) optimized so as to reduce the nonspecific effects.

E—Blocking antibodies RA synoviocytes seeded onto a 96-well plate (1×10⁴ cells/well) were preincubated (2 h, 37° C.) with the anti-IL-17RA monoclonal antibodies (10 μg/ml), alone or with etanercept (10 μg/ml). The RA synoviocytes were then stimulated with the IL-17A or IL-17F (50 μg/ml), alone or in combination with TNF-α (0.5 ng/ml) for 36 h.

F—ELISA (Enzyme Linked ImmunoSorbent Assay) RA synoviocytes seeded onto a 96-well plate (1×10⁴ cells/well) were stimulated with IL-17A or IL-17F (50 ng/ml), alone or in combination with TNF-α (0.5 ng/ml) for 12, 24 or 36 h. IL-6 and IL-8 were quantified in the culture supernatants by ELISA using reagents from eBiosciences (San Diego, Calif.) and Diaclone (Besancon, France), respectively.

G—Reverse transcription and quantitative PCR (Polymerase Chain Reaction) After 2 h of serum deprivation, the RA synoviocytes seeded into 6-well plates (5×10⁵ cells/well) were stimulated for 1, 3, 6 or 12 h with IL-17A or IL-17F (50 ng/ml), alone or in combination with TNF-α (0.5 ng/ml). The total RNA was isolated by TRIzol extraction (Invitrogen Life Technologies) according to the recommended instructions. The nucleic acids were quantified by spectrophotometry at 260 nm (SmartSpec™3000, BIO-RAD, Hercules, Calif.). 1 μg of nucleic acids was used for the reverse transcription (ThermoScript™ RT-PCR System, Invitrogen Life Technologies). Briefly, the total RNA was denatured (65° C., 5 min) in the presence of oligo(dT) primers. The reverse transcription was then carried out in the presence of dNTPs (0.5 mM), RNase OUT (40 U/μl), dithiothreitol (0.01 M) and reverse transcriptase (10 U/μl; ThermoScript™). After incubation for 60 min at 50° C., the reaction was stopped (85° C., 5 min) and the complementary DNAs (cDNAs) obtained were diluted (1:10) in distilled water. A volume of 10 μl of cDNA solution was used per amplification.

The primers specific for IL-6, for Il-8/CXCL8, for GAPDH and for HPRT1 come from Search-LC (Heidelberg, Germany), whereas the primers specific for IL-17RA (Genbank accession No.: NM_{—}014339) and for IL-17RC (Genbank accession No.: NM_{—}153461) were synthesized by the company Eurogentec (San Diego, Calif.). IL-17RA sense: SEQ ID No.1 5′-AGACACTCCA GAACCAATTC C-3′, IL-17RA antisense: SEQ ID No.2 5′-TCTTAGAGTT GCTCTCCACC A-3′, IL-17RC sense: SEQ ID No.3 5′-ACCAGAACCT CTGGCAAGC-3′, IL-17RC antisense: SEQ ID No.4 5′-GAGCTGTTCA CCTGAACACA-3′. The amplification reactions were carried out using a Light Cycler (Roche Molecular Biochemicals, Meylan, France) with the specific reagents (LightCycler FastStart DNA Sybr Green I kit, Roche Molecular Biochemicals). A standard amplification protocol was used to amplify the IL-6, the IL-8/CXCL8, the IL-17RA, the GAPDH and the HPRT1 (45 amplification cycles: denaturation at 96° C., hybridization from 68° C. to 58° C., amplification at 72° C.), whereas the amplification of the IL-17RC transcripts was carried out with an optimized protocol (45 amplification cycles: denaturation at 99° C., hybridization from 68° C. to 58° C., amplification at 72° C.). The number of copies of mRNA of interest was standardized using GAPDH and HPRT1.

H—Western blotting. The expression of IL-17RA and of IL-17RC was measured by Western blotting using murine antibodies directed against human IL-17RA and goat antibodies directed against human IL-17RC (R & D systems). The protein concentration was measured using a BCA kit. 80 μg of total proteins were separated on an SDS-10% polyacrylamide gel and transferred onto a Hybond-C extra nitrocellulose membrane (Millipore, Bedford, Mass.). The membranes were incubated in series with antibodies directed against actin (Chemicon, Hampshire, United Kingdom), IL-17RA and IL-17RC. The blots were scanned, and the densitometric data for IL-17RA and for IL-17RC were standardized using actin and expressed in arbitrary units (AU) (Image Gauge software, version 3.46).

I—Immunohistochemistry. Fragments of synovial membrane were fixed in 10% formaldehyde. After paraffin embedding, 4-μm sections of the samples were cut, mounted on slides, and deparaffinized (OTTIX Plus, DiaPath, Martinengo, Italy). Antigen unmasking was carried out by incubation in citrate buffer (pH 6) for 40 minutes at 99° C. The endogenous peroxidase activity was blocked with hydrogen peroxide at 3% for 5 minutes, before incubation for one hour with the primary antibody: 10 μg/ml of murine monoclonal anti-IL-17A antibody (IgG2b), 10 μg/ml of murine monoclonal anti-IL-17RA antibody (IgG1) or 10 μg/ml of goat polyclonal anti-IL-17RC antibody. On the control sections, the same concentrations of an irrelevant antibody were used (mouse IgG2b, mouse IgG1 or goat serum, respectively). After washing, the sections were incubated with biotinylated anti-mouse or anti-goat antibodies for 15 minutes, followed by incubation with streptavidin-peroxidase for 15 minutes, and then chromogenic 3,3′-diaminobenzidine (DAB) solution (DAKO, Glostrup, Denmark). The sections were then counter-stained with Mayer's hematoxylin.

J—DNA chips. 5 μg of RNA from total peripheral blood (31 RA patients and 19 healthy volunteers) and 2 μg of RNA obtained from experiments carried out on RA synoviocytes were analyzed using HG-U133A chips (Affymetrix, Santa Clara, Calif., USA) (IVT labeling protocol). The amount of RNAs was studied using RNA 6000 nano chips and the Agilent 2100 bioanalyzer (Agilent Technologies, Waldbronn, Germany). The total RNA was used to prepare double-stranded cDNAs containing a T7 promoter sequence. cRNAs were synthesized and labeled with biotinylated ribonucleotides (GeneChip IVT Labeling Kit, Affymetrix). Fragmented cRNAs were hybridized on HG-U133A chips (22 283 probe sets). The chips were washed and labeled using an FS450 fluidics station (Affymetrix) (EukGE-WS2v4 protocol), and then scanned with the Agilent G2500A scanner. The statistical analysis were generated using the Affymetrix analytical software (MAS 5.0).

Statistical analysis The protein levels were expressed as mean±SEM. The levels of mRNA of interest were standardized with the GAPDH mRNA levels and the data were expressed as induction relative to the untreated control situations. The statistical values of the differences were determined using the Dunnett test and the differences resulting in a value P<0.05 were considered to be statistically significant.

Results

A—Effects of IL-17A and IL-17F on IL-6 secretion by synoviocytes derived from patients suffering from rheumatoid arthritis (RA) In order to compare the effect of IL-17A and of IL-17F on IL-6 secretion, synoviocytes derived from patients suffering from RA (RA synoviocytes) were stimulated with increasing concentrations of IL-17A or of IL-17F (from 0.1 to 100 ng/ml), and the IL-6 secretion was measured in the culture supernatants by ELISA. After treatment for 48 h, IL-17F induced IL-6 secretion in a dose-dependent manner (FIG. 1A). The cooperative effects of IL-17A or of IL-17F with TNF-α were studied. RA synoviocytes were stimulated for 48 h with IL-17A or IL-17F (50 ng/ml), alone or in combination with a suboptimal concentration of TNF-α (0.5 ng/ml), and the IL-6 secretion was assayed by ELISA (FIG. 1B). The RA synoviocytes treated with IL-17A (50 ng/ml), IL-17F (50 ng/ml) or TNF-α (0.5 ng/ml), alone, induced, respectively, 5.9±0.4, 2.5±0.1 or 13.6±1.6 ng/ml of IL-6. In the presence of TNF-α, the IL-6 secretion induced by IL-17A or IL-17F was synergistically increased (43.4±1.6, P<0.05 and 30.8±13.7, P<0.05). IL-17F induced levels comparable to IL-17A in the presence of TNF-α.

B—Effects of IL-17A and IL-17F on the expression of IL-6 mRNA and IL-8 mRNA derived from patients suffering from RA The effect of IL-17A and of IL-17F was studied on RA synoviocytes by analyzing the effect induced on the levels of IL-6 mRNA and IL-8 mRNA (FIG. 2). RA synoviocytes were stimulated with IL-17A or IL-17F (50 ng/ml), alone or in combination with TNF-α. After treatment for 12 h, the total RNA was extracted and the amounts of IL-6 mRNA and IL-8 mRNA were measured by quantitative PCR. IL-17A, IL-17F or TNF-α, alone, significantly increased the amounts of IL-6 mRNA (induction factor relative to the situation without treatment: 15.8±4.1, 2.7±0.5 and 17±2.8, respectively, P<0.05) (FIG. 2A). In the presence of TNF-α, IL-17A and IL-17F synergistically increased the amounts of IL-6 mRNA (induction factor relative to the situation without treatment: 175.9±57.7 and 72.3±30.7, respectively, P<0.05).

The inventors also compared the ability of IL-17A and of IL-17F to regulate the expression of IL-8, a chemokine involved in neutrophil recruitment. After stimulation with IL-17F for 12 h, the amounts of IL-8 mRNA were increased (induction factor relative to the situation without treatment: 3.2±0.4 for IL-17F, 47.1±21.7 for IL-17A, P<0.05) (FIG. 2B). In the presence of TNF-α, the amounts of IL-8 mRNA induced by IL-17F were comparable to those induced by IL-17A (induction factor relative to the situation without treatment: 829±358.1 for IL-17A plus TNF-α, and 584.8±275 for IL-17F plus TNF-α, P>0.05).

C—Effect of the knockdown of IL-17RA and IL-17RC receptors on IL-6 secretion and IL-8 secretion induced by IL-17A. As demonstrated by quantitative PCR, basal expression of the two receptors was observed in the RA synoviocytes, with amounts of IL-17RA mRNA 35 times higher than the amounts of IL-17RC mRNA (P<0.05). The functional contribution of these two receptors to the biological effects of IL-17A and of IL-17F was subsequently studied using the interfering RNA technique (iRNA). The transfection of RA synoviocytes with IL-17RA iRNAs (0.5 μg) or IL-17RC iRNAs (0.05 μg) induced, 24 h later, a mean reduction of 80% in the amounts of IL-17RA mRNA and of 62% in the amounts of IL-17RC mRNA, respectively (FIG. 3A). The RA synoviocytes were then stimulated with TNF-α (0.5 ng/ml), IL-17A or IL-17F (50 ng/ml) for 12 h, 48 h after transfection with the IL-17RA iRNAs, the IL-17RC iRNAs or with the iRNAs used as negative control (siCONTROL). The IL-6 was then assayed in the supernatants by ELISA. As represented in FIG. 3B, the transfection with the IL-17RA iRNAs or with the IL-17RC iRNAs significantly decreased the IL-6 secretion induced by IL-17A (mean±SEM after transfection with the IL-17RA iRNAs or the IL-17RC iRNAs compared with the siCONTROL iRNAs: 1.3±0.2 or 1.6±0.3 compared with 3±0.9 respectively, P<0.05). The specific involvement of the two receptors in the effects of IL-17A was, moreover, supported by the absence of significant effect of the IL-17RA iRNAs or of the IL-17RC iRNAs on the IL-6 secretion induced by TNF-α (mean±SEM after transfection with the IL-17RA iRNAs or the IL-17RC iRNAs, compared with the siCONTROL iRNAs: 2.9±0.7 and 2.7±0.7 compared with 2.5±4 respectively, P>0.9).

D—Effect of the Knockdown/Blocking of IL-17RA and IL-17RC Receptors by Inhibitors (Interfering RNA or Specific Antibody) on IL-6 Secretion Induced by IL-17A or IL-17F, in the Presence of TNF-α

TNF-α, a cytokine overexpressed in the rheumatoid synovial tissue, is involved in the physiopathology of RA. The analysis of the contribution of the IL-17RA and IL-17RC receptors, in an inflammatory context modeled, in vitro, by the presence of IL-17A or of IL-17F (50 ng/ml) and of TNF-α (0.5 ng/ml), was studied.

iRNA: The RA synoviocytes were transfected with IL-17RA iRNA, IL-17RC iRNA or siCONTROL iRNA, and then stimulated with IL-17A or IL-17F, alone or in combination with TNF-α, for 36 h.

The decrease in the expression of the IL-17RA or IL-17RC receptors alone had no significant effect on IL-6 secretion induced by IL-17A in the presence of TNF-α (mean±SEM after transfection with the IL-17RA iRNAs or the IL-17RC iRNAs compared with the siCONTROL iRNAs: 33.6±5.1 and 36.1±4.1 compared with 32.6±6.6; P>0.9), whereas the simultaneous decrease in the expression of the IL-17RA and IL-17RC receptors induced a 20% reduction in secreted IL-6. Moreover, the IL-6 secretion induced by IL-17F in the presence of TNF-α did not vary significantly after transfection with the IL-17RA iRNAs or the IL-17RC iRNAs (mean±SEM after transfection with the IL-17RA iRNAs and the IL-17RC iRNAs compared with the siCONTROL iRNAs, 12.8±4.2 and 11.3±4.1 compared with 16.9±1.7; P>0.9), whereas the cotransfection with the iRNAs specific for the two receptors decreased the IL-6 secretion (mean±SEM after transfection with the IL-17RA iRNAs and the IL-17RC iRNAs compared with the siCONTROL iRNAs: 12.1±0.5 compared with 16.9±1.7; P<0.05).

Specific antibodies: The inventors compared the effect of inhibiting the IL-17RA and IL-17RC receptors using interfering RNA with an extracellular blocking approach by means of specific antibodies. The inventors thus tested the effect of neutralizing antibodies directed against the IL-17RA and/or IL-17RC receptors. These antibodies were tested alone or in combination with etanercept®, a soluble form of the TNF-α receptor type II (p75), commonly used clinically. RA synoviocytes were preincubated with the inhibitor(s) for 2 h, and then stimulated for 36 h with IL-17A or IL-17F (50 ng/ml), alone or in combination with TNF-α (0.5 ng/ml).

The assaying of IL-6, used to determine the effect of the various inhibitors, made it possible to demonstrate that the blocking of IL-17RA or of IL-17RC with antibodies had a significant effect in the presence of IL-17A alone (mean±SEM in the presence of anti-IL-17RA antibody or of anti-IL-17RC antibody compared with the situation without inhibitors: 2.0±0.7 and 3.0±1.1 compared with 5.2±1.8; P<0.05), whereas the blocking thereof was insufficient to significantly reduce the effect of IL-17A in the presence of TNF-α (mean±SEM in the presence of anti-IL-17RA antibody or of anti-IL-17RC antibody compared with the situation without inhibitors: 34.7±6.8 and 40.6±9.1 compared with 37.6±6.3; P>0.9).

The inventors demonstrated that blocking the IL-17RA or IL-17RC receptors reduced IL-6 secretion induced by IL-17F in the presence of TNF-α.

Finally, the combination of the two antibodies, anti-IL-17RA and anti-IL-17RC, reduced by 33% the IL-6 secretion induced by IL-17A in the presence of TNF-α (mean±SEM in the presence of anti-IL-17RA antibody and of anti-IL-17RC antibody compared with the situation without inhibitors: 25.3±8.6 compared with 37.6±6.3; P<0.05) and by 19% the IL-6 secretion induced by IL-17F in the presence of TNF-α.

The inventors also observed a significant effect of Etanercept® on the IL-6 secretion induced by TNF-α alone or in the presence of IL-17A or of IL-17F (mean±SEM in the presence of Etanercept® compared with the situation without inhibitors: induced by TNF-α alone, 1.2±0.2 compared with 5.4±1.1, P<0.05; induced by TNF-α plus IL-17A, 8.5±4.0 compared with 37.6±1.1, P<0.05; induced by TNF-α plus IL-17F, 1.9±0.5 compared with 15.2±4.3, P<0.05). Finally, the simultaneous blocking of the IL-17RA or IL-17RC receptors and TNF-α dramatically reduced the IL-6 secretion induced by IL-17A or IL-17F in the presence of TNF-α (FIG. 4).

E—IL-17RA and IL-17RC are Overexpressed in the Total Peripheral Blood of RA Patients.

The inventors also examined the expression of the mRNAs encoding IL-17RA and IL-17RC in the peripheral blood by means of DNA chips (FIG. 5A). A significant increase in the expression of mRNAs encoding IL-17RA and IL-17RC was observed in the RA patients (n=40) compared with the healthy volunteers (HV) (n=19) (median IL-17RA: 262.5 versus 237.2; P<0.005 and median IL-17RC: 50.5 versus 44.3; P<0.0005).

The expression of IL-17RA and of IL-17RC was also analyzed at the protein level by Western blotting (FIG. 5B). As at the mRNA level, a significantly higher expression of IL-17RA was found compared with IL-17RC (median IL-17RA versus IL-17RC at the mRNA level (n=59) or protein level (n=9): 253.7 versus 48.24; P<0.0001 or 22.77 versus 2.45; P<0.0001, respectively).

F—IL-17RA and IL-17RC are Expressed in the Synovial Membrane Derived from Patients Suffering from RA

The inventors also analyzed the expression of IL-17RA and of IL-17RC by immunohistochemistry in the synovial membrane derived from patients suffering from RA and from arthrosis (OA), and showed that the two receptors were expressed in a diffuse and superimposable manner in the RA synovial membrane (FIG. 6A, B, C, D). A similar diffuse labeling was observed in the OA synovial membrane (FIG. 6E, F). This diffuse expression confirms the expression of these receptors, in the stromal cells and the infiltrating cells. As a control, the inventors analyzed the expression of IL-17A, which was detected in the lymphocyte infiltrates, in cells with a plasma-cell morphology (inset of FIG. 6D).

1. A method of manufacturing a medicament for inhibiting, preventing or treating rheumatoid arthritis, comprising:
providing at least one interleukin-17F inhibitor and/or of at least one IL-17 receptor inhibitor.

2. A method of manufacturing a medicament for inhibiting, preventing or treating rheumatoid arthritis, comprising:
providing at least one interleukin-17F inhibitor and/or of at least one IL-17 receptor inhibitor in combination with a treatment against TNF-alpha.

3. The method of claim 1, wherein said IL-17 receptor is IL-17 receptor A or IL-17 receptor C.

4. The method of claim 2, wherein said treatment against TNF-alpha is chosen from etanercept, Infliximab® and adalimumab.

5. The method of claim 4, wherein said treatment against TNF-alpha is etanercept.

6. The method of claim 1, wherein the interleukin-17F inibitor is an antibody directed against IL-17F, and/or the inhibitor of said IL-17 receptor is an antibody directed against the IL-17 receptor.

7. The method of claim 1, wherein the interleukin-17F inhibitor is an interfering RNA against IL-17F, and/or the inhibitor of said IL 17 receptor is an interfering RNA against the IL-17 receptor.

8. A pharmaceutical composition comprising, as active ingredient, at least one interleukin-17F inhibitor and/or at least one IL-17 receptor inhibitor in combination with a pharmaceutically appropriate carrier.

9. The pharmaceutical composition as claimed in claim 8, wherein it also comprises a treatment against TNF-alpha.

10. The composition as claimed in claim 8, wherein said IL-17 receptor is IL-17 receptor A or IL-17 receptor

11. The composition as claimed in claim 9, wherein said treatment against TNF-alpha is chosen from etanercept, Infliximab® and alimumab.

12. The composition as claimed in claim 11, wherein said treatment against TNF-alpha is etanercept.

13. The composition as claimed in claim 8, wherein the interleukin-17F inhibitor is an antibody directed against IL-17F, and/or the inhibitor of said IL-17 receptor is an antibody directed against the IL-17 receptor.

14. The composition as claimed in claim 8, wherein the interleukin-17F inhibitor is an interfering RNA against IL-17F, and/or the inhibitor of said IL-17 receptor is an interfering RNA against the IL-17 receptor.

15. A method for inhibiting, preventing or treating rheumatoid arthritis, comprising:
providing the pharmaceutical composition of claim 8.

16. An in vitro method for determining, on the basis of a biological sample, the early diagnosis of rheumatoid arthritis, the response of a patient suffering from rheumatoid arthritis to a treatment directed against a cytokine involved in the inflammatory process of the disease, and/or the monitoring of the response of a patient suffering from rheumatoid arthritis to a treatment directed against a cytokine involved in the inflammatory process of the disease, over time, characterized in that the expression of the gene encoding IL-17A, IL-17F, IL-17RA and/or IL-17RC is determined.

17. The in vitro method as claimed in claim 16, according to which the measurement of the expression of the gene encoding IL-17A, IL-17F, IL-17RA and/or IL-17RC comprises the following steps:
a) biological material is extracted from the biological sample,
b) the biological material is brought into contact with at least one reagent specific for the gene encoding IL-17A, IL-17F, IL-17RA and/or IL-17RC;
c) the expression of the gene encoding IL-17A, IL-17F, IL-17RA and/or IL-17RC is determined.

18. A method for determining the early diagnosis of rheumatoid arthritis, the response of a patient suffering from rheumatoid arthritis to a treatment directed against a cytokine involved in the inflammatory process of the disease, and/or the monitoring of the response of a patient suffering from rheumatoid arthritis to a treatment directed against a cytokine involved in the inflammatory process of the disease, over time, comprising:
providing at least one reagent specific for the gene encoding IL-17A, IL-17F, IL-17RA and/or IL-17RC.

19. A kit for making an early diagnosis of rheumatoid arthritis, for giving a prognosis for the response of a patient suffering from rheumatoid arthritis to a treatment directed against a cytokine involved in the inflammatory process of the disease, and/or for giving a prognosis for the monitoring of the response of a patient suffering from rheumatoid arthritis to a treatment directed against a cytokine involved in the inflammatory process of the disease, over time, comprising at least one reagent specific for the gene encoding IL-17A, IL-17F, IL-17RA and/or IL-17RC.


SEQUENCE LISTING (4 sequences)

SEQ ID NO: 1 - DNA, 21 bp, Homo sapiens
agacactccagaaccaattcc

SEQ ID NO: 2 - DNA, 21 bp, Homo sapiens
tcttagagttgctctccacca

SEQ ID NO: 3 - DNA, 19 bp, Homo sapiens
accagaacctctggcaagc

SEQ ID NO: 4 - DNA, 20 bp, Homo sapiens
gagctgttcacctgaacaca